(19) United States
(12) Patent Application Publication
(10) Pub. No.: US 2024/0350804 A1
(43) Pub. Date: Oct. 24, 2024

(54) SYSTEMS AND METHODS FOR INCREASING THE SELECTIVITY AND EFFICACY OF SACRAL NEUROMODULATION

(71) Applicant: Duke University, Durham, NC (US)

(72) Inventors: Warren Grill, Durham, NC (US); Daniel Marshall, Durham, NC (US)

(21) Appl. No.: 18/639,063

(22) Filed: Apr. 18, 2024

Related U.S. Application Data
(60) Provisional application No. 63/460,672, filed on Apr. 20, 2023.

(51) Int. Cl. A61N 1/36; A61N 1/05; G16H 20/40

(52) U.S. Cl. CPC A61N 1/36062; A61N 1/0551; A61N 1/36007; A61N 1/36071; A61N 1/36107; A61N 1/3615; A61N 1/36171; A61N 1/36178; A61N 1/36185; G16H 20/40

(57) ABSTRACT

A method of treating or preventing at least one symptom associated with a disease or condition in a subject in need thereof. The method comprising: stimulating a sacral nerve in the subject with an electrode according to a set of stimulation parameters; wherein the set of stimulation parameters is based on a three-dimensional model. The three-dimensional model includes a pelvic organs model, a sacral nerve model, and an electrode model. Electrode designs for stimulating a sacral nerve in a subject in need thereof.

### RELATED APPLICATIONS

This application claims priority to and the benefit of U.S. Provisional Patent Application No. 63/460,672 filed Apr. 20, 2023, which is incorporated herein by reference in its entirety for all purposes.

### FIELD

The present disclosure provides systems and methods relating to neuromodulation. In particular, the present disclosure provides systems and methods for increasing the selectivity and efficacy of sacral neuromodulation.

### BACKGROUND

Overactive Bladder (OAB) is a debilitating condition that in the U.S. affects 16.0% of men and 16.9% of women. When medication fails to treat OAB, sacral nerve stimulation (SNS) has proven to be an effective and safe treatment option, with up to 90% of patients seeing >50% symptom improvement. While SNS has been in use since its FDA approval in 1997, an understanding of the underlying mechanisms is lacking, and programming of device outputs is often a trial-and-error process. Further, the effectiveness may be limited by the dynamic range between activation of neural elements that produce the targeted inhibition of symptoms and the onset of stimulation-evoked side effects. Thus, there is an ongoing need for improved treatments.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Embodiments of the present disclosure include a method of treating or preventing at least one symptom associated with a disease or condition in a subject in need thereof. The method comprising: stimulating a sacral nerve in the subject with an electrode according to a set of stimulation parameters. The set of stimulation parameters is selected based on a three-dimensional model; wherein the three-dimensional model includes a pelvic organs model, a sacral nerve model, and an electrode model.

In some embodiments, the method further comprises placing the electrode in a location suitable to deliver electrical stimulation to the sacral nerve.

In some embodiments, the location is at least partially determined from the three-dimensional model.

In some embodiments, the disease or condition comprises overactive bladder, urge urinary incontinence, urinary urgency frequency, underactive bladder, urinary retention, fecal incontinence, constipation, pelvic pain, and/or sexual dysfunction.

In some embodiments, at least one symptom comprises urinary frequency, urinary urgency, nocturia, urge incontinence, feeling of not being empty, small voided volumes, straining to void, pain, or any combination thereof.

In some embodiments, the sacral nerve model includes a plurality of fascicles and epineurium based on histology nerve cross sections and/or imaging data.

In some embodiments, the three-dimensional model includes the electrode model positioned at least partially within a foramen in a sacrum of the pelvis model.

In some embodiments, the set of stimulation parameters includes one or more of: stimulation pulse amplitude, stimulation pulse duration, stimulation waveform shape, stimulation pulse repetition rate, and temporal pattern of stimulation pulses.

Embodiments of the present disclosure include an electrode for stimulating a sacral nerve in a subject in need thereof, the electrode comprising: a) a plurality of contacts, wherein center-to-center spacing between adjacent contacts is within a range of about 6 mm to about 18 mm; b) a plurality of circumferentially segmented contacts; c) a plurality of tines, wherein a contact is positioned at a terminal end of each of the plurality of tines; and/or d) a shaft and a loop portion with a first contact at a first end of the loop portion and a second contact at a second end of the loop portion.

In some embodiments, the electrode comprises a plurality of contacts, wherein the center-to-center spacing between adjacent contacts is within a range of about 12 mm to about 16 mm.

In some embodiments, the electrode comprises at least three circumferentially segmented contacts.

In some embodiments, the electrode comprises a plurality of tines extending radially away from a centerline of the electrode, and each of the plurality of tines includes a plurality of contacts along a length of the tine.

In some embodiments, the electrode comprises a shaft and a loop portion extending from the shaft; wherein the loop portion includes a first contact at a first end of the loop portion, a second contact at a second end of the loop portion, and an intermediate contact positioned between the first contact and the second contact.

In some embodiments, the loop portion is a first loop portion and the electrode further comprises a second loop portion extending from the shaft.

In some embodiments, the first loop portion is positioned at a terminal end of the shaft and the second loop portion is axially spaced from the first loop portion along the shaft.

Embodiments of the present disclosure include a method of selecting a desired lead position to treat or prevent at least one symptom associated with a disease or condition in a subject in need thereof, the method comprising: generating a three-dimensional model of a pelvis and a sacral nerve based on imaging from a subject; adding an electrode model to the three-dimensional model in a candidate position relative to the sacral nerve; simulating neural activation of the sacral nerve in the three-dimensional model for the electrode model in the candidate position; moving the electrode model in the three-dimensional model to an alternative position relative to the sacral nerve; simulating neural activation of the sacral nerve in the three-dimensional model for the electrode model in the alternative position; and selecting the desired lead position from one of the candidate position and the alternative position based on simulated neural activation.

In some embodiments, the method further comprises adding an alternative electrode model to the three-dimensional model; simulating neural activation of the sacral nerve in the three-dimensional model for the alternative electrode model in a plurality of positions relative to the sacral nerve; and selecting a desired lead design from one of the electrode model and the alternative electrode model based on simulated neural activation.

In some embodiments, simulating neural activation of the sacral nerve in the three-dimensional model includes simulating a plurality of candidate lead configurations and determining activation of nerve fibers in a plurality of fascicles.

Embodiments of the present disclosure include a method of selecting a desired lead configuration to treat or prevent at least one symptom associated with a disease or condition in a subject in need thereof, the method comprising: generating a three-dimensional model of a pelvis and a sacral nerve based on imaging from a subject; adding an electrode model to the three-dimensional model in a position relative to the sacral nerve; wherein the position is based on imaging from the subject; simulating neural activation of the sacral nerve in the three-dimensional model for a plurality of candidate lead configurations of the electrode model; and selecting the desired lead configuration from one of the plurality of candidate lead configurations based on simulated neural activation.

In some embodiments, the method further comprises programming a pulse generator to deliver electrical stimulation to the subject based on the desired lead configuration.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 illustrates a sacral nerve stimulation (SNS) system including a waveform generator and a lead.

FIG. 2 illustrates histology and segmentation for the third sacral nerve root in accordance with one embodiment of the present disclosure. In particular, a histological slice from 2 mm distal to the nerve root ganglion.

FIG. 3A-3D illustrates geometrical models in accordance with one embodiment of the present disclosure. In particular, geometrical models: A) pelvic geometry imported from into Simpleware ScanIP; B) boundary of the geometrical model; C) finite element mesh of (A) generated in Simpleware ScanIP; and D) S3 nerve root and lead geometry created in COMSOL and placed inside the third sacral foramen.

FIG. 4A-4E illustrates A) datasets with segmented nerve trajectories matched to sacrum morphology have different overall threshold values, but relative threshold between fascicles change little: B) Visible Human Female (VH) cryosections; C) segmented S1-S4; D) VH CT scans; E) segmented sacrum.

FIG. 5 illustrates S3 nerve root and lead geometry created in COMSOL and placed inside the third sacral foramen of the imported mesh.

FIG. 6A-6C illustrates modeling of electric currents physics: A) results of curvilinear coordinates simulation in the S3 endoneurium to determine material anisotropy; B) Vₑ from an electric currents simulation on the epineurium and bone surface; and C) Vₑ from an electric currents simulation shown along fiber paths.

FIG. 7 illustrates a method of creating computational models of SNS using pelvic morphology and histology of the third sacral nerve root, and quantifying the response of the model nerve fibers to SNS.

FIG. 8A-8C illustrates the effects of varied contact configurations in accordance with one embodiment of the present disclosure. Effects of varied contact configurations: A) all possible contact configurations; B) data in (A), organized by the separation between active contacts; and C) current flow lines for distant and adjacent contacts.

FIG. 9A-9D illustrates effects of contact on S3 activation using the reference model: A) Electrode placement relative to the S3 cross section plane; B) Threshold results and fascicle selectivity ratios for all possible contact configurations; C) Threshold results for all contact configurations, organized by the distance separating the active contacts. Points are colored according to the fascicle colors in B; D) Stimulation results using two point sources in an infinite, homogeneous, anisotropic medium, at various inter-source spacings, angles, and distances from the fiber (diameter=10 μm).

FIG. 10A-10E illustrates effects of varying lead positioning in accordance with one embodiment of the present disclosure. Effects of varying lead positioning: A) mediolateral rotations; B) superior-inferior rotations; C) proximal-distal shifts; D) mediolateral shifts; and E) superior-inferior shifts.

FIG. 11A-11F illustrates effects of lead positioning and orientation on S3 thresholds: A) Cartoon of electrode placement in the third sacral foramen; the center of rotation was the midpoint between electrodes 1 and 2; B) transverse shifts; C) electrode-fiber distance shifts; D) longitudinal shifts; E) transverse rotations; F) electrode-fiber distance rotations.

FIG. 12 is a perspective view of a lead design having four contact lead with increased contact spacing.

FIG. 13A is a perspective view of a lead design having segmented leads.

FIG. 13B is a cross-sectional view of the lead design of FIG. 13A.

FIG. 13C is a perspective view of the lead design of FIG. 13A positioned relative to the sacral nerve.

FIG. 13D illustrates the contacts of lead design of FIG. 13A positioned relative to the sacral nerve.

FIG. 13E illustrates simulation threshold results for different contact configurations of the lead design of FIG. 13A.

FIG. 14A is a perspective view of a lead design having a plurality of tines.

FIG. 14B is a plan view of the lead design of FIG. 14A.

FIG. 14C is a perspective view of the lead design of FIG. 14A positioned relative to the sacral nerve.

FIG. 14D illustrates the contacts of the lead design of FIG. 14A positioned relative to the sacral nerve.

FIG. 14E illustrates simulation threshold results for different contact configurations of the lead design of FIG. 14A.

FIG. 15A is a perspective view of a lead design having a plurality of loop portions.

FIG. 15B is a plan view of the lead design of FIG. 15A.

FIG. 15C is a perspective view of the lead design of FIG. 15A positioned relative to the sacral nerve.

FIG. 15D illustrates the contacts of the lead design of FIG. 15A positioned relative to the sacral nerve.

FIG. 15E illustrates simulation threshold results for different contact configurations of the lead design of FIG. 15A.

### DETAILED DESCRIPTION

The present disclosure provides systems and methods relating to neuromodulation. In particular, the present disclosure provides systems and methods for increasing the selectivity and efficacy of sacral neuromodulation.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Pain” generally refers to the basic bodily sensation induced by a noxious stimulus, received by bare nerve endings, characterized by physical discomfort (e.g., pricking, throbbing, aching, etc.) and typically leading to an evasive action by the individual. As used herein, the term pain also includes chronic and acute neuropathic pain. The term “chronic neuropathic pain” refers to a complex, chronic pain state that wherein the nerve fibers themselves may be damaged, dysfunctional or injured. These damaged nerve fibers send incorrect signals to other pain centers. The impact of nerve fiber injury includes a change in nerve function both at the site of injury and areas around the injury. The term “acute neuropathic pain” refers to self-limiting pain that serves a protective biological function by acting as a warning of on-going tissue damage. Acute neuropathic pain is typically a symptom of a disease process experienced in or around the injured or diseased tissue.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

The term “effective amount” or “therapeutically effective amount” refers to an amount sufficient to effect beneficial or desirable biological and/or clinical results.

The systems described herein can be implemented in hardware, software, firmware, or combinations of hardware, software and/or firmware. In some examples, the systems described in this specification may be implemented using a non-transitory computer readable medium storing computer executable instructions that when executed by one or more processors of a computer cause the computer to perform operations. Computer readable media suitable for implementing the systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system described in this specification may be located on a single device or computing platform or may be distributed across multiple devices or computing platforms.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. OVERVIEW

Overactive Bladder (OAB) is a debilitating condition characterized by frequent and uncomfortable urges to urinate, and in many cases, urinary incontinence. In the U.S., OAB affects 16% of men and 17% of women. OAB significantly impacts the quality of life (QoL) of individuals, leading to social, psychological, and financial burdens. The economic burden of OAB each year is estimated to be $24.9 billion. Many cases of OAB are resistant (refractory) to behavioral and pharmacological interventions, leading to a need for therapies which are effective in this population.

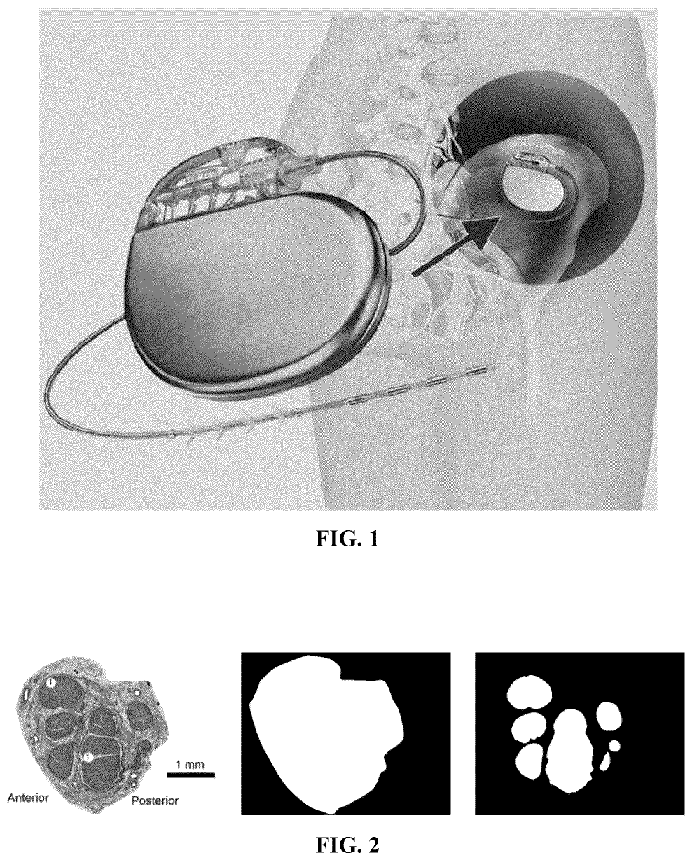

Sacral nerve stimulation (SNS) has proven an effective treatment for refractory (treatment-resistant) overactive bladder. Sacral nerve stimulation involves the electrical stimulation of the sacral nerve roots, typically the third sacral nerve (S3), via a lead implanted in the sacral foramen. The lead has four cylindrical contacts along its length, that can be configured in a bipolar or monopolar fashion. The lead is connected to an implantable pulse generator (IPG) placed in the fatty tissue of the buttocks. Two devices are currently available, the Medtronic InterStim (FIG. 1) and Axonics systems.

While effective for many, SNS has seen little innovation in stimulation parameters and lead designs since its inception. The lack of a comprehensive understanding of the mechanism underlying SNS contributes to the trial-and-error approach currently employed in SNS programming, limiting its potential efficacy and patient reach. Barriers to advancing SNS include a limited understanding of its operational mechanisms, particularly the role of somatic afferent activation in its therapeutic effects. While it's speculated that SNS aids in restoring the normal function of the pontine micturition center, the precise neural interactions remain elusive. This knowledge gap not only hampers the development of more effective stimulation paradigms but also restricts the ability to mitigate side effects associated with motor efferent activation. It is not known whether enabling increased stimulation amplitudes through avoidance of motor efferent activation can increase efficacy.

In response to these challenges, this disclosure provides a comprehensive computational model of SNS, incorporating three-dimensional pelvic anatomy alongside detailed nerve geometry. Computational modeling emerges as a formidable tool to bridge these gaps, offering the potential to study the complex interactions between electrical stimulation and nerve responses. However, existing models of SNS suffer from significant limitations, particularly in terms of anatomical precision and the biophysical properties of nerve tissues. These shortcomings have prevented a thorough understanding of the intricacies involved in SNS therapy. By exploring the effects of various model parameters and contact configurations, this study aims to elucidate the biophysical underpinnings of SNS efficacy, paving the way for improved therapeutic strategies and a broader understanding of sacral neuromodulation in treating Overactive Bladder.

One aspect of the present disclosure provides a computational model of SNS using three-dimensional pelvic morphology. The disclosure describes the use of computational modeling to analyze nerve responses to stimulation and develop approaches to expand the dynamic range. Specifically, the computational model can be used to calculate the relative activation of different neural elements, the use of the computational model to quantify, analyze, and optimize the patterns of neural activation produced by different electrode geometries and stimulation parameters, and electrode geometries that enable more selective and effective sacral neuromodulation. A model of the stimulation lead was parameterized and placed alongside a three-dimensional nerve geometry with fully modeled tissues. The response of biophysically realistic axon models placed within the nerve geometry was quantified.

Additionally, the effects of model parameters and electrode geometry on activation thresholds in the third sacral nerve (S3) root were quantified.

Computational models of SNS using pelvic morphology and histology of the third sacral nerve root (S3) are disclosed herein. The response to SNS of the model nerve fibers are quantified, and new lead designs are provided to enable selective activation of S3. The present disclosure may be used in the field of OAB treatment using neuromodulation and can be used to: plan SNS device interventions; aid in clinical programming of an implanted SNS device; evaluate novel electrode designs and stimulation paradigms; and improve efficacy of SNS technologies through novel contact placement designs, lead positioning methods, and lead designs. In some embodiments, other applications of the systems and methods disclosed herein include use in developing SNS for fecal incontinence, chronic constipation, urinary retention, pelvic pain, and/or sexual dysfunction. In some embodiments, other applications of the systems and methods disclosed herein include use in developing SNS for stimulation of nerves other than S3, e.g., the S1, S2, S4 and/or S5 sacral spinal nerves. In some embodiments, other applications of the systems and methods disclosed herein include use in developing spinal nerve stimulation for stimulation of spinal nerves other than the sacral nerves, e.g., the cervical, thoracic, and/or lumbar spinal nerves.

As disclosed herein, a SNS computational model employs 3D pelvic morphology to: calculate the relative activation of different neural elements; quantify, analyze, and optimize neural activation patterns formed by different electrode configurations and stimulation parameters; and develop new electrode designs that enable greater selectivity and efficacy of sacral neuromodulation. As discussed further herein, increasing contact spacing in the SNS lead to decreased activation thresholds, and thereby increase stimulation efficiency and device lifetime as shown in the left panel of the figure below: The disclosure also provides methods of improved lead positioning using activation data from lead contact and pre-operative imaging. Novel lead designs are also disclosed herein to increase SNS efficacy and reduce stimulation side effects by increasing control of the post-implantation current flow direction and thus, removing a large portion of wasted lateral current.

In one aspect the disclosure provides an anatomically realistic 3D finite element model of the human pelvic morphology. In one aspect, the disclosure provides a method of improved lead placement using activation data from lead contact and pre-operative imaging such as an MRI scan. In one aspect, the disclosure provides model derived novel designs for SNS lead and contact placement within lead.

### 3. METHODS

Morphology and Model Geometry. Implementation and simulation of an anatomically realistic three-dimensional finite element model of the human pelvis, sacral nerves, and surrounding tissues is described herein. The geometry of the model can include morphological information from published literature, histological material, and/or imaging, including MRI, CT, X-ray, or other imaging modalities. The model includes a representation of an electrode or electrodes that deliver electrical stimulation. The output of the model is the distribution of electrical potentials, as well as the spatial paths of current flow, resulting from applied stimulation.

The outputs of the finite element model are coupled to non-linear cable models of myelinated axons and/or unmyelinated axons to calculate the neural response to applied electrical stimulation. Alternatively, the non-linear cable models may be replaced with approximations including linear cable models, activation functions, or machine learning implementations trained to estimate the response of non-linear nerve fibers, to quantify and/or characterize the nerve responses to applied electrical signals. The diameter and positions of the nerve fibers are based on human nerves. The output of this stage of the model is a measure of nerve fiber activation, modulation, or block as a function of stimulation parameters, for a range of nerve fiber diameters (i.e., fiber types) and positions. The model will, for various stimulation parameters, quantify activation, modulation, or block of different nerve fiber populations including sensory axons and motor axons innervating different organs or other surrounding nerves. The output can be displayed as input-output curves of nerve fiber activation, modulation, or block for targeted and non-targeted populations as a function of stimulation parameters.

The integrated finite element-nerve fiber model can be used to plan device interventions. Planning can include the calculation of the patterns of nerve fiber activation, modulation, or block (and therapeutic dynamic ranges) for different electrode geometries, electrode locations, and stimulation parameters in patient-specific versions of the models or general forms of the models. These simulations can be used to select the appropriate intervention for specific patients based upon interventions that produce the desired pattern of nerve fiber activation, modulation, or block, the avoidance of nerve fiber activation, modulation, or block, and/or the desired therapeutic dynamic range or other performance criteria.

The integrated finite element-nerve fiber model can be used to assist in clinical programming of a device implanted for clinical therapy. Device programming can include selection of the active electrode contacts and their polarity. Device programming can include the selection of the parameters of the applied electrical signal including signal amplitude, signal pulse duration, signal waveform shape, signal pulse repetition rate, and the temporal pattern of signal application. The outputs of the model can include calculation of the patterns of nerve fiber activation, modulation, block, therapeutic dynamic range, or other performance criteria for different electrode geometries, electrode locations, and electrical signal parameters in patient-specific or general forms of the models. These model outputs can be displayed and used to select the appropriate parameters that produce the desired pattern of nerve fiber activation, modulation, block, therapeutic dynamic range, or other performance criteria. Algorithms can be coupled to the model to determine automatically, through optimization, the appropriate parameters that produce the desired pattern of nerve fiber activation, modulation, block, therapeutic dynamic range, or other performance criteria.

The integrated finite element-nerve fiber model can be used to develop and conduct model-based evaluation of novel electrode designs and stimulation paradigms. For example, the designs can increase the dynamic range between activation of the targeted nerve fibers that produce the desired changes in symptoms and activation of non-targeted nerve fibers that produce unwanted side effects. The designs can reduce the sensitivity of nerve fiber activation to electrode positioning. The designs can increase the efficiency of nerve activation, increasing efficacy and battery life. The designs can achieve other specified performance objectives. The model can be coupled to an optimization algorithm to determine specific attributes of the electrode design or applied electrical signal parameters that minimize a cost function to achieve the desired device performance. Performance can include the desired pattern of nerve fiber activation, modulation, block, therapeutic dynamic range, or other performance criteria. Novel electrode designs that will increase the effectiveness of sacral neuromodulation in treating symptoms and reduce side effects of stimulation include changes in the number, size, and spacing of electrode contacts and the use of segmented (i.e., directional) electrode contacts that span only a sub-segment of the lead circumference. The electrode can include additional insulation to shield current from flowing in specific directions. The electrode can constitute a multiple-contact electrode array with different contacts programmed at different polarities and/or amplitudes to achieve field steering and direct the stimulation to, for example, occur in the targeted neural elements and to avoid stimulation in the non-targeted neural elements. The multiple-contact electrode array can be programmed with different contacts set to different polarities and/or amplitudes to produce the desired pattern of nerve fiber activation, modulation, block, therapeutic dynamic range, or other performance criteria.

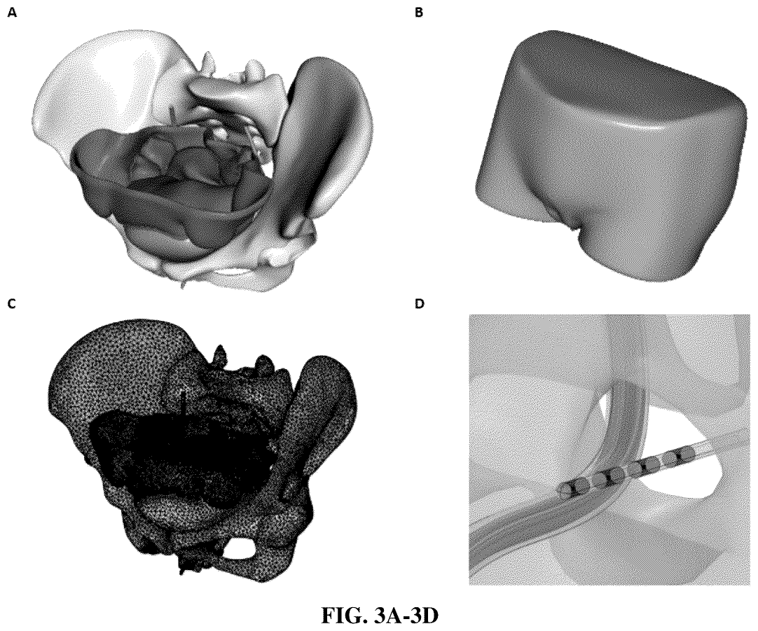

A finite element model is created for sacral nerve stimulation using, in some embodiments, published pelvis segmentation and S3 cross sections. With reference to FIG. 2, the sacral nerve histology is segmented from following the methods described herein. The segmentation is inflated by 20% to account for shrinkage during histological processing. In some embodiment, a Nikon's NIS-Elements AR software (v5.02.01, Build 1270, Nikon Instruments Inc.) is used to segment published sacral nerve histology (FIG. 2). In some embodiments, publicly available segmentation of the female pelvis is used to build a three-dimensional model (FIGS. 3A and 3B), including the following structures: the sacrum, coccyx, iliac bones, colon, uterus, ovaries, ovarian tubes, the bladder, bladder wall, ureters, and the peritoneum. Small structures are excluded which are distant from the S3 foramen. In some embodiments, the model is converted from a 3D-PDF to an STL using Tetra4D (Tetra4D, Bend, OR). If data on the scale of the model is available, the pelvis model is scaled to the average size of the female pelvis (as determined by measuring the sacral body width). The geometry is imported and meshed in, for example, Simpleware ScanIP (version T-2022.03, Synopsys, Mountain View, CA). In some embodiments, a finite element mesh is created using linear tetrahedra with “coarseness” set to −5. A path was created for the sacral nerve that followed the sacral spine, coursed anteriorly through the foramen, and reached the bladder. In some embodiments, the file is exported as a NASTRAN mesh for use in COMSOL.

The nerve path coordinates, pelvic organ NASTRAN mesh, and skin is then imported into COMSOL Multiphysics (FIG. 3C), generating a nerve cross-section from segmented histology, and using COMSOL's “sweep” operation to create three-dimensional nerve geometry along the imported path (FIG. 3D). In some embodiments, a work plane is created interpolation curves used to build a cross section of the nerve and fascicles (endoneurium). COMSOL's “sweep” operation is used to create three-dimensional nerve geometry by sweeping our cross section along the imported path (FIG. 3D). Because nerve models using a single cross-section have highly homogeneous intra-fascicle responses, a single three-dimensional fiber line is created along the centroid of each fascicle. A model of a four-contact electrode array is parameterized and instrumented according to ideal surgical positioning. The space between the pelvic structures and skin is meshed, as well as the lead and nerve in COMSOL.

In some embodiments, sacrum segmentations and 3D nerve paths are created using the publicly available Visible Human Datasets. For each dataset, the 70 mm high resolution TIFF cryosection images are downloaded, as well as the CT scan images. XnView is used to convert from the CT scan images to TIFFs and imported the images into Simpleware ScanIP. Using the paint tool, we marked a series of slices along each axis (xy, yz, zw) and used the 3D Wrap tool to create a segmentation of the sacrum. We then created a segmentation of the tissue inside the sacrum using a series of slice interpolations (painting a series of slices along the xy axis and using the interpolation tool to create a 3D object) merged together with a boolean union. We subtracted this from the 3D wrapped sacrum segmentation to create a final sacrum segmentation, to which we applied Simpleware's smart mask smoothing. For the paths of the sacral nerve roots, we imported the cryosection images into Simpleware ScanIP. The visible human male dataset had large jumps in its transverse positioning, and we used motion correction to smooth out the jumps. The image stack had a circular object which moved along with the pelvis. We manually segmented this object, then calculated for each slice the centroid position. We shifted each successive image so that the centroid lay over the first image's centroid, resulting in a smoothed dataset. For both datasets, the cryosection images were scaled incorrectly in the x and y directions. We corrected the scaling by measuring the width of the grayscale reference card which appeared in each image. The actual size of the card is 8 mm in length, and we rescaled the cryosection image stack so that the length was correct. For each sacral nerve root, we painted slices along the path of the nerve in the xy plane, and then used an interpolation operation to create a 3D object of the nerve. For the left S3 root, we used the “convert to centrelines” tool to obtain a set of coordinates along the centroid of the nerve, which we exported for use in COMSOL. Finally, to match the CT and cryosection datasets to each other, we used Simpleware's dataset registration tools (manual) to line the two up. We then meshed the sacrum segmentation and exported the mesh as an STL file for use in COMSOL.

In some embodiments, models are created in COMSOL from the visible human datasets using the same methods as the reference model, except the sacrum geometry is remeshed using an “adapt” and converted the resultant mesh to a geometry. These models did not include a representation of any geometry other than the sacrum. For the skin boundary, we instead used a cylinder with the top and bottom grounded. For both models there were slight intersections between our generated S3 geometry and the sacrum, thus we imported the intersecting S3 geometry into Simpleware ScanIP, dilated it by 2 pixels in the x and y directions, subtracted the dilated mask from the sacrum, and re-exported the sacrum to COMSOL.

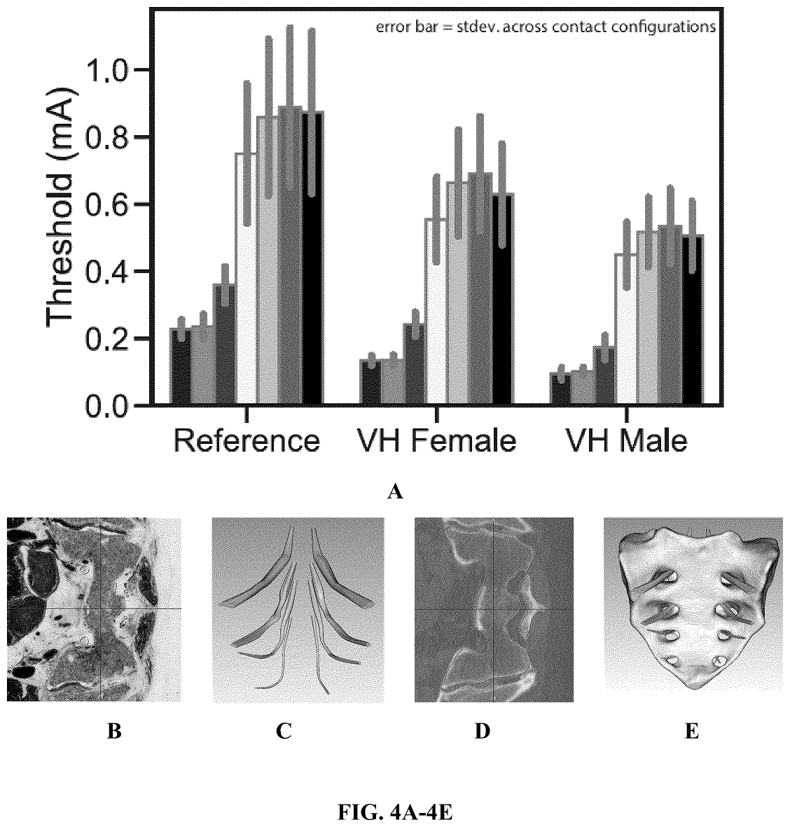

With reference to FIG. 4A, datasets with segmented nerve trajectories matched to sacrum morphology have different overall threshold values, but relative threshold between fascicles change little.

To examine the impact of sacral morphological variability on our model, we created a segmentation of the sacra and sacral nerves from the visible human datasets and compared threshold results using the same S3 cross section as the reference model. We matched the average electrode fiber distance across all models. A 2 factor ANOVA showed that while there was a main effect of model on thresholds, there was no interaction between fascicle and model, indicating that the relative thresholds were the same between all models. Variability in sacral morphology may impact overall threshold values through increased or decreased impedance (due to the size and shape of the foramen), however, it has little effect on the relative threshold values between fascicles.

FIG. 4B illustrates a Visible Human Female (VH) cryosections. FIG. 4C illustrates a segmented S1-S4. FIG. 4D illustrates a VH CT scan. FIG. 4E illustrates a segmented sacrum.

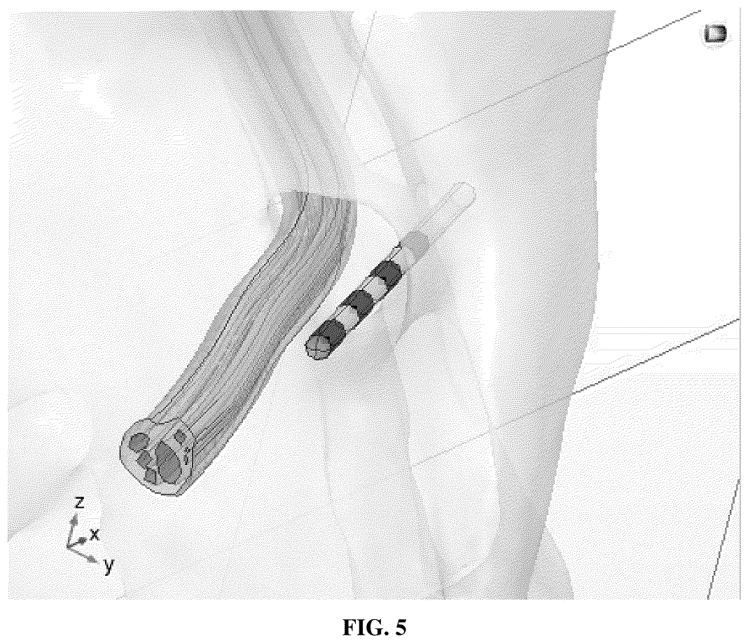

With reference to FIG. 5, the S3 nerve root and lead geometry created in COMSOL and placed inside the third sacral foramen of the imported mesh.

Electric Currents and NEURON Simulations. We applied a ground condition to the top and bottom surfaces of the skin and assigned material properties to the geometry. We assigned the volume enclosed by the skin which was not represented by any of the included organ geometry as fat. As the endoneurium is anisotropic in the longitudinal direction, we solved a curvilinear coordinates diffusion simulation in the endoneurial geometry to define a new vector system for applying non-cartesian (i.e., a spatially varying vector field) anisotropy to the endoneurium. We modeled each fascicle with a sheet resistance based on the perineurium thickness relationship described in. We placed a point current source at the center of each cylindrical electrode contact.

Using the principle of superposition, we could represent the summation of electric fields from different contacts by the linear sum of fields from individual contacts. Thus, we solved four finite element models for each geometrical representation, each with one point current source active, and the others inactive. We solved Laplace's equations for each model using a quasi-static approximation (i.e., stationary solution). We extracted the potentials from the line along each fascicle's centroid, using 100× element refinement and polynomial preserving derivative recovery to obtain high longitudinal resolution.

Using these “super-sampled” potentials, we created models of 13 μm (representing a typical Aa motor fiber) MRG fibers and linearly resampled our extracted potentials at the center coordinate of each model compartment. We weighted the potentials obtained from COMSOL in various combinations (to test different contact configurations) and scaled them in time using a typical clinical biphasic waveform with 210 us per phase. We applied these weighted, time-scaled potentials to each fiber model and solved the simulations using backward Euler integration in NEURON 8.2. Using a bisection search algorithm, we determined the stimulation amplitude threshold for fiber activation with 1% resolution. We analyzed and plotted the threshold data using Python 3.10.

After creating our geometry and finite element mesh in COMSOL, we assigned material properties (electrical conductivity) to the geometry as given in Table 1. We assigned the conductivity of fat to the volume enclosed by the skin which was not represented by any of the organ geometries. As the endoneurium is anisotropic in the longitudinal direction, we solved a curvilinear coordinates diffusion simulation in the endoneurial geometry to define a new vector system for applying anisotropic (i.e., a spatially varying vector field) conductivity to the endoneurium. We used a “diffusion” model, with the rostral ends of the fascicles as the inlet and the caudal ends as the outlets. This simulation provided a new coordinate system for assigning conductivity; we assigned the higher conductivity along the first basis vector (along the path of the fascicles) and the lower conductivity to the other two basis vectors.

We modeled each fascicle's perineurium as a sheet resistance on its boundary based on the perineurium thickness to fascicle size relationship. We applied a ground condition to the top and bottom surfaces of the skin. We placed a point current source at the center of each cylindrical InterStim™ contact. We solved one simulation for each contact, assigning the active contact's point current source to 1 mA, and all other contacts to 0 mA. For each model, we solved Laplace's equations using a stationary solution. Because nerve models using a single cross-section have highly homogeneous intra-fascicle responses, we created one three-dimensional fiber path along the centroid of each fascicle. We extracted the potentials from each fascicle, for each simulation, using 100× element refinement and polynomial preserving derivative recovery to obtain high longitudinal resolution (referred to as “supersample potentials”).

We used fiber modeling to create biophysical simulations of extracellular stimulation of MRG model fibers. We ran our simulations using NEURON 8.2 and Python 3.10. For each fiber path, we created models of 13 μm (representing a typical Aa motor fiber and sensory fibers). We linearly resampled our extracted supersampled potentials at the center coordinate of each fiber compartment. Using the principle of superposition, we could represent the summation of electric potentials from different active contacts by the linear sum of fields from individual contacts. Thus we weighted the potentials obtained from COMSOL in various combinations (to test different contact configurations) and then scaled them in time using a typical clinical biphasic waveform with 210 us per phase. We applied these weighted, time-scaled potentials to each fiber model and solved the simulations using backward Euler integration. Using a bisection search algorithm, we determined threshold stimulation amplitude for fiber activation with 1% resolution (difference between top and bottom search bounds).

TABLE I
MATERIAL AND TISSUE CONDUCTIVITIES
USED IN THE FINITE ELEMENT MODELS.

| Material | Conductivity (S/m) |
|---|---|
| polyurethane | 10\hat{ }−14 |
| platinum | 9.43 × 10\hat{ }6 |
| endoneurium | {1/6, 1/6, 1/1.75} |
| epineurium | 1/6.3 |
| perineurium | 1/1149 |
| bladder wall | 3.15E−01 |
| urine | 2.44E+00 |
| ureter | 2.32E−01 |
| uterus | 3.91E−01 |
| peritoneum | 7.92E−02 |
| ovaries | 3.66E−01 |
| uterine tubes/uterus | 3.91E−01 |
| bone | 0.02 |
| colon | 0.01 |

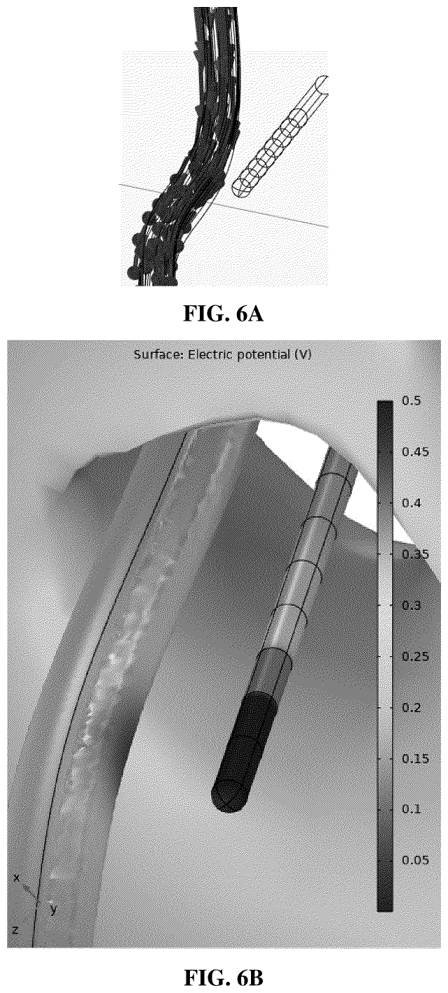

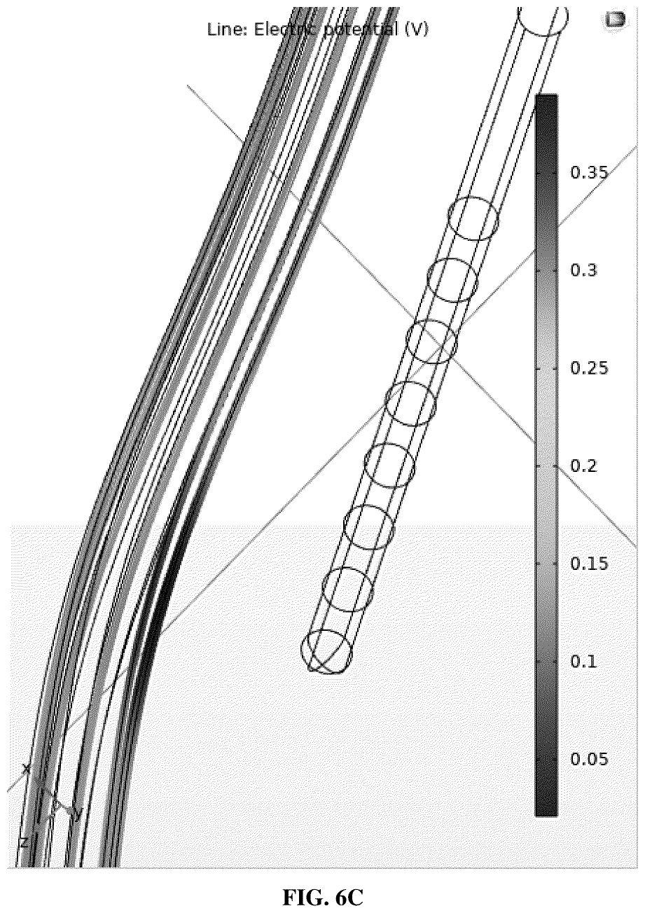

With reference to FIG. 6A, the results of curvilinear coordinates simulation in the S3 endoneurium. FIG. 6B illustrates Vₑ from an electric currents simulation on the epineurium and bone surface. FIG. 6C illustrates Vₑ from an electric currents simulation shown along the fiber paths.

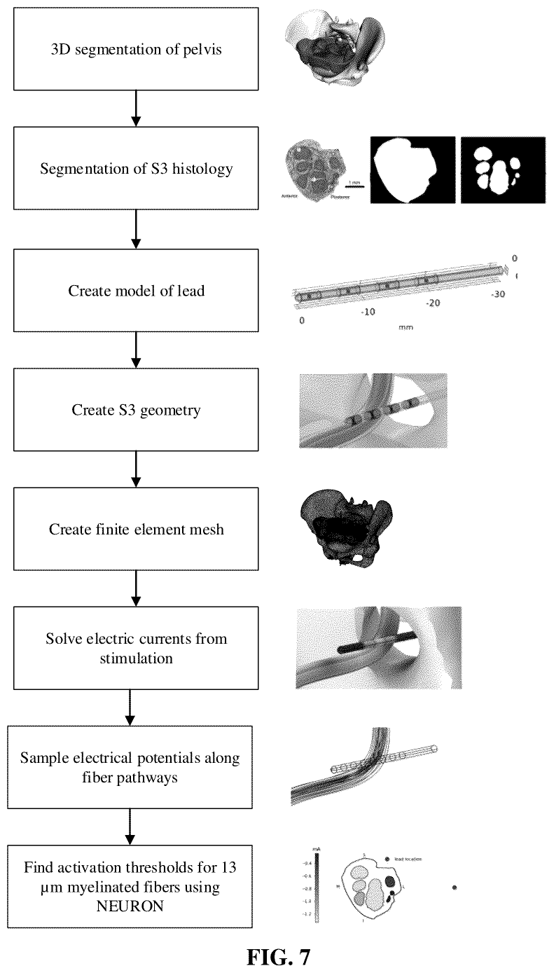

With reference to FIG. 7, a method is illustrated including steps of: 3D segmentation of pelvis; segmentation of S3 histology; create model of lead; create S3 geometry; Create finite element mesh; solve electric currents from stimulation; sample electrical potentials along fiber pathways; and find activation thresholds. FIG. 7 illustrates a method of creating computational models of SNS using pelvic morphology and histology of the third sacral nerve root, and quantifying the response of the model nerve fibers to SNS.

Embodiments of the present disclosure include a method of treating or preventing at least one symptom associated with a disease or condition in a subject in need thereof, the method comprising: stimulating a sacral nerve in the subject with an electrode according to a set of stimulation parameters; wherein the set of stimulation parameters is selected based on a three-dimensional model; wherein the three-dimensional model includes a pelvic organs model, a sacral nerve model, and an electrode model.

In some embodiments, the method further comprises placing the electrode in a location suitable to deliver electrical stimulation to the sacral nerve.

In some embodiments, the location is at least partially determined from the three-dimensional model.

In some embodiments, the disease or condition comprises overactive bladder, urge urinary incontinence, urinary urgency frequency, underactive bladder, urinary retention, fecal incontinence, constipation, pelvic pain, and/or sexual dysfunction.

In some embodiments, at least one symptom comprises urinary frequency, urinary urgency, nocturia, urge incontinence, feeling of not being empty, small voided volumes, straining to void, pain, or any combination thereof.

In some embodiments, the sacral nerve model includes a plurality of fascicles and epineurium based on histology nerve cross sections and/or imaging data.

In some embodiments, the three-dimensional model includes the electrode model positioned at least partially within a foramen in a sacrum of the pelvis model.

In some embodiments, the set of stimulation parameters includes one or more of: stimulation pulse amplitude, stimulation pulse duration, stimulation waveform shape, stimulation pulse repetition rate, and temporal pattern of stimulation pulses.

Embodiments of the present disclosure include a method of selecting a desired lead position to treat or prevent at least one symptom associated with a disease or condition in a subject in need thereof. The method comprising: generating a three-dimensional model of a pelvis and a sacral nerve based on imaging from a subject; adding an electrode model to the three-dimensional model in a candidate position relative to the sacral nerve; simulating neural activation of the sacral nerve in the three-dimensional model for the electrode model in the candidate position; moving the electrode model in the three-dimensional model to an alternative position relative to the sacral nerve; simulating neural activation of the sacral nerve in the three-dimensional model for the electrode model in the alternative position; and selecting the desired lead position from one of the candidate position and the alternative position based on simulated neural activation.

In some embodiments, the method further comprises adding an alternative electrode model to the three-dimensional model; simulating neural activation of the sacral nerve in the three-dimensional model for the alternative electrode model in a plurality of positions relative to the sacral nerve; and selecting a desired lead design from one of the electrode model and the alternative electrode model based on simulated neural activation.

In some embodiments, simulating neural activation of the sacral nerve in the three-dimensional model includes simulating a plurality of candidate lead configurations and determining activation of nerve fibers in a plurality of fascicles.

Embodiment of the present disclosure include a method of selecting a desired lead configuration to treat or prevent at least one symptom associated with a disease or condition in a subject in need thereof. The method comprises: generating a three-dimensional model of a pelvis and a sacral nerve based on imaging from a subject; adding an electrode model to the three-dimensional model in a position relative to the sacral nerve; wherein the position is based on imaging from the subject; simulating neural activation of the sacral nerve in the three-dimensional model for a plurality of candidate lead configurations of the electrode model; and selecting the desired lead configuration from one of the plurality of candidate lead configurations based on simulated neural activation.

In some embodiments, the method further comprises programming a pulse generator to deliver electrical stimulation to the subject based on the desired lead configuration.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

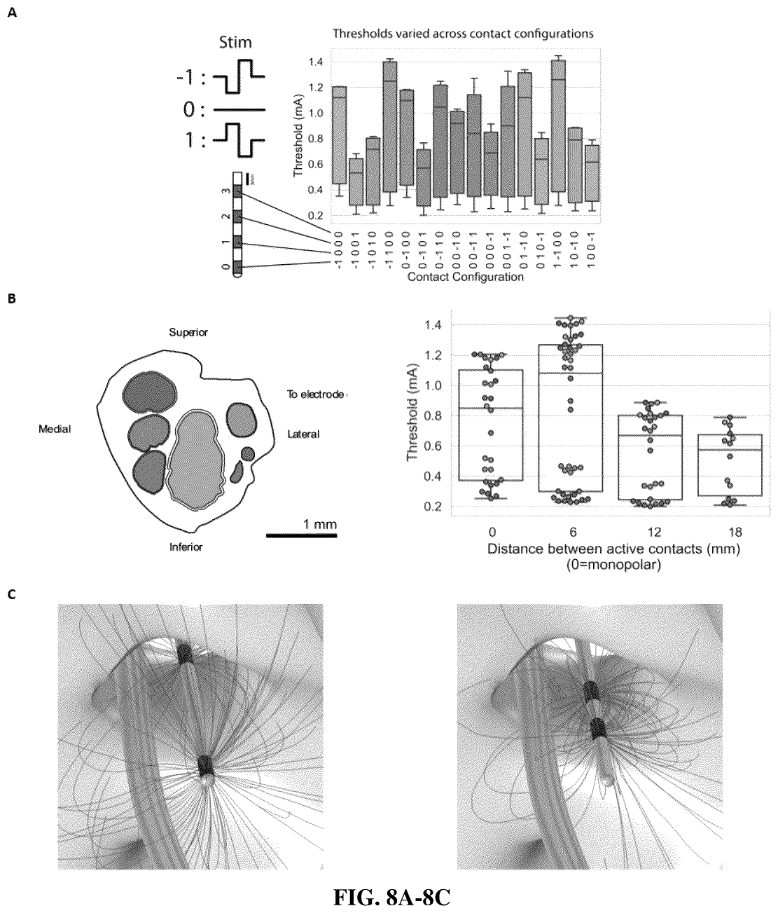

Lead Configuration. With reference to FIG. 8A, the choice of active contacts can have a marked effect on the activation thresholds of S3 fibers. Lower activation thresholds increase battery life, and stimulation efficacy is typically associated with contacts that are readily able to produce motor and sensory responses. Current clinical practice typically uses adjacent active contacts; our data show that changing clinical recommendations to prioritize increased contact spacing may lead to lower activation thresholds, and thus more effective stimulation and longer-lasting devices (FIG. 8B). Conversely, the separation between thresholds of a given fascicle decreases at greater contact spacing, meaning that while tighter contact spacing may decrease thresholds, it may also open the door to more selective activation of fibers responsible for therapeutic effects of SNS. FIG. 8C shows current flow lines, more of which reach the target nerve when contacts are spaced further apart. Additionally, it can be observed that much of the current escapes laterally (to the right) and is wasted. Lead designs that allow for radial selectivity would likely confer a great benefit over the current clinical designs.

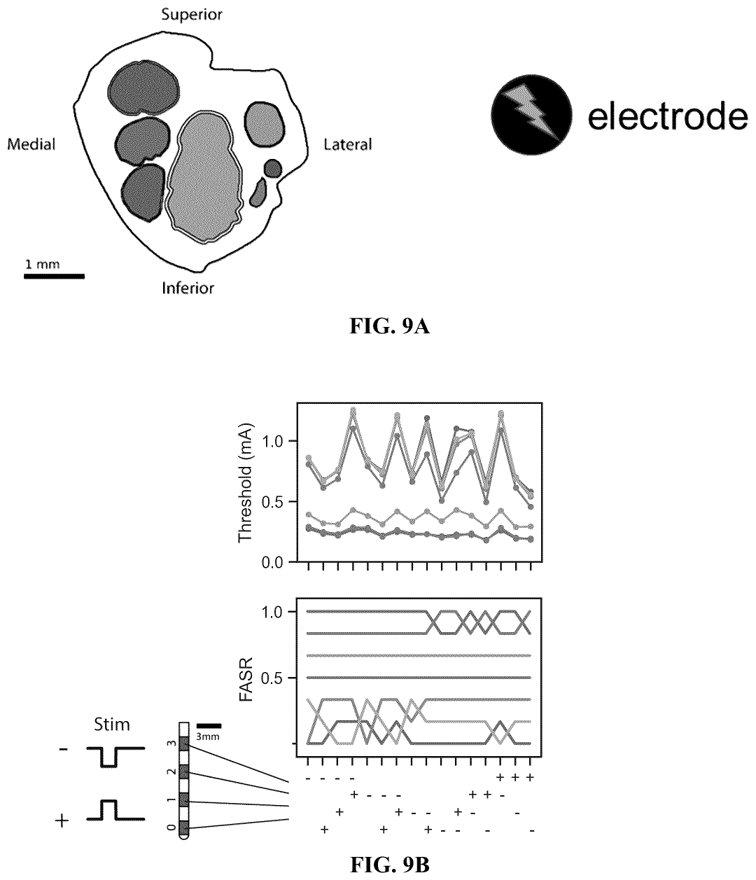

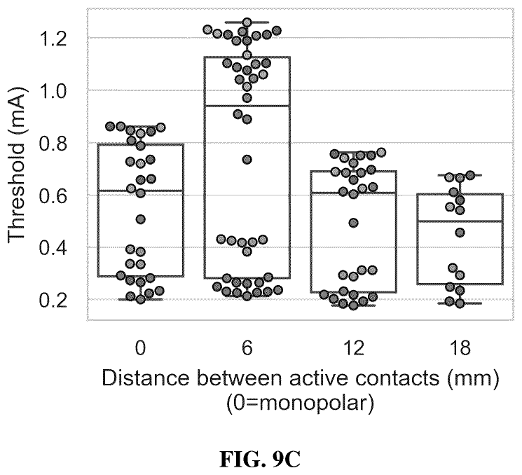

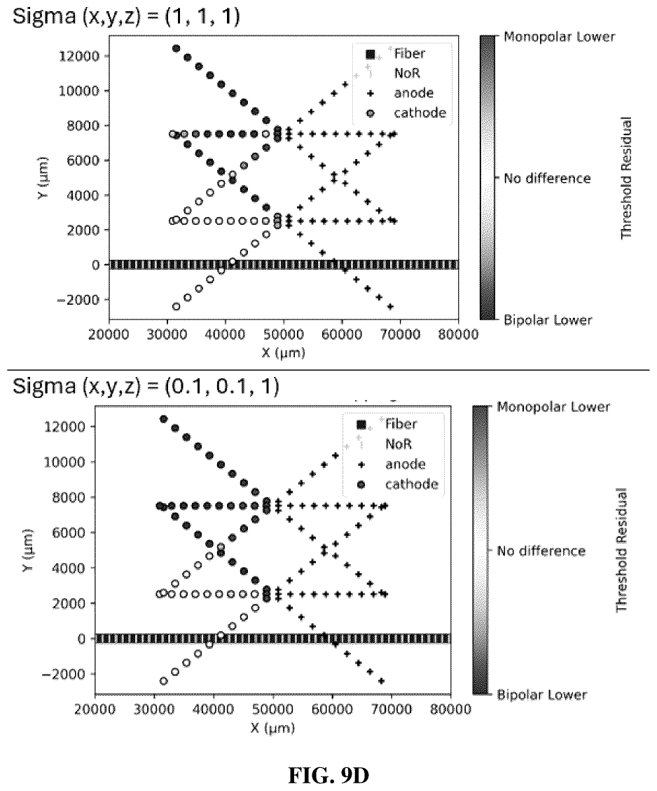

FIG. 9A-9D illustrates effects of contact on S3 activation using the reference model: A) Electrode placement relative to the S3 cross section plane; B) Threshold results and fascicle selectivity ratios for all possible contact configurations; C) Threshold results for all contact configurations, organized by the distance separating the active contacts. Points are colored according to the fascicle colors in B; D) Stimulation results using two point sources in an infinite, homogeneous, anisotropic medium, at various inter-source spacings, angles, and distances from the fiber (diameter=10 μm).

The effect of contact configuration on thresholds in S3 is examined by simulating all possible monopolar and bipolar contact configurations. Contact configuration had a large influence on activation thresholds (FIG. 9A); these changes had nearly the dynamic range of moving the lead+−2 mm towards/away from the nerve (FIG. 9C). We calculated fascicle selectivity ratios across the fascicles in each configuration. While thresholds varied greatly across contact configurations, the order in which fascicles were recruited remained relatively stable. We grouped each contact configuration according to the distance separating the active contacts (0 for monopolar stimulation) (Error! Reference source not found.). Configurations with directly adjacent contacts (6 mm apart) had higher thresholds than monopolar configurations, while configurations with greater spacing (12 mm, 18 mm) had lower thresholds. The furthest possible spacing had the lowest thresholds. Increasing contact spacing lowers thresholds to a point; we verified that high enough contact spacings (using a longer lead) thresholds begin to increase thresholds. The result of lower thresholds for further bipolar contact spacings matches experimental data in sheep. However, two studies in humans have found that monopolar stimulation has lower thresholds than bipolar stimulation. To explore this mismatch, we conducted simulations using a fiber in an infinite, homogeneous, isotropic medium. We placed points sources centered over the middle of a long 10 μm fiber. We varied the distance of the centroid between the point sources, the distance between the point sources, and the angle between the points sources and the fiber. We found that, as with the full pelvis model, thresholds for closely spaced bipolar configurations were lower than monopolar thresholds, and further spacing caused bipolar thresholds to be lower than monopolar thresholds.

### Example 2

Lead Positioning. Lead positioning primarily effects thresholds through changes in electrode-fiber distance. Lead positioning is thought to be vital for the efficacy of SNS, but it can often be unclear where the lead is located in relation to the nerve during surgery. S3 is not visible in intraoperative CT images, and placement is guided by the sacral foramen alone. Placement is verified by sensory and motor fiber activation as well as intraoperative flouroscopy, however, our data suggest that relative activation data may be able to determine the positioning of the lead with respect to S3 on a per-contact basis.

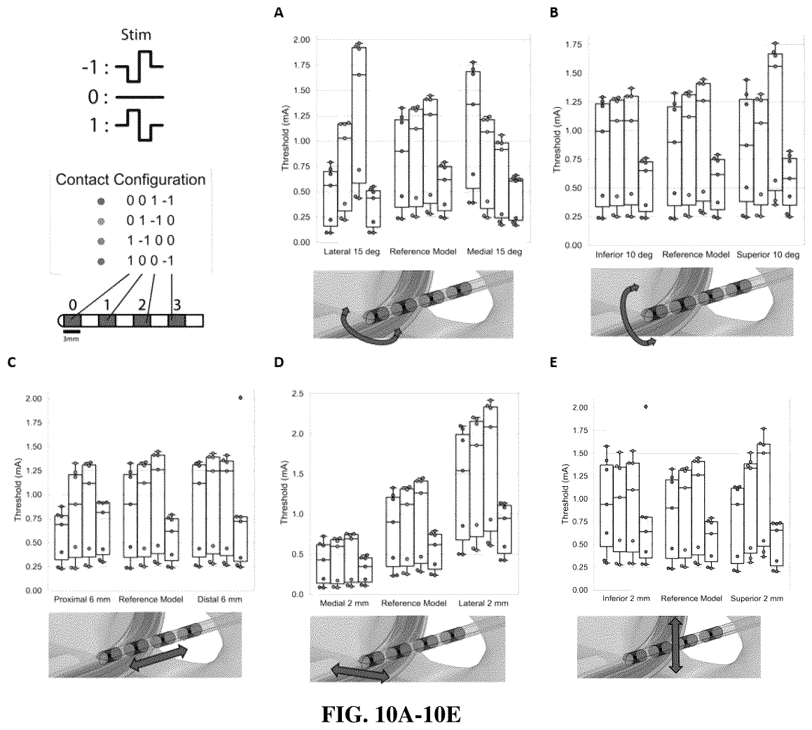

With reference to FIG. 10, lead positioning can have a marked effect on the relationship between thresholds for different contact configurations, particularly where the positioning alters electrode-fiber distance. A powerful addition to the proposed technique would be pre-operative imaging of the nerve (prior work has shown a 3T MRI is sufficient to segment S3). This imaging could also be used to inform a patient-specific model of optimal lead positioning for the activation of S3 fibers using our modeling techniques.

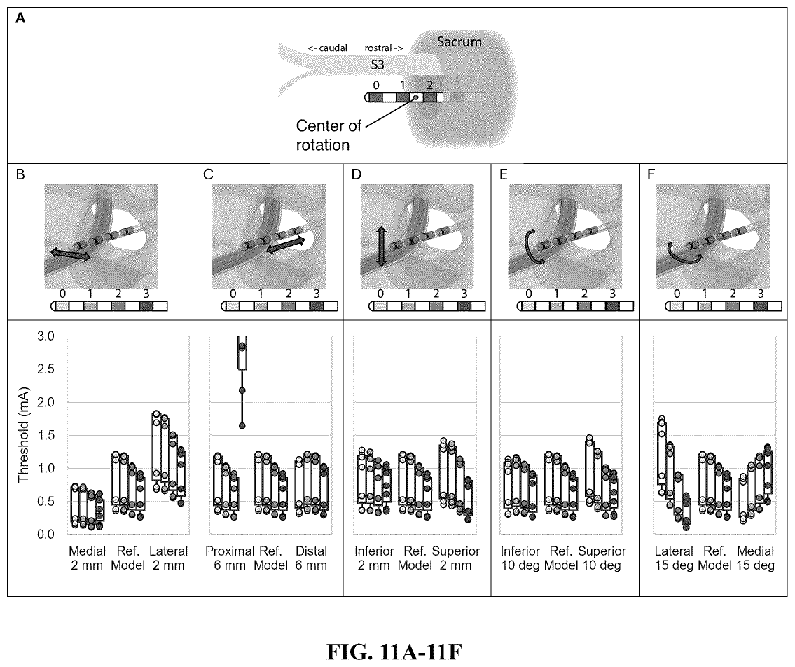

FIG. 11A-11F illustrates effects of lead positioning and orientation on S3 thresholds: A) Cartoon of electrode placement in the third sacral foramen; the center of rotation was the midpoint between electrodes 1 and 2; B) transverse shifts; C) electrode-fiber distance shifts; D) longitudinal shifts; E) transverse rotations; F) electrode-fiber distance rotations.

We investigated the effects of lead positioning on activation thresholds within S3, using monopolar cathodic stimulation on each contact. As would be expected, we found that electrode fiber distance was the primary contributor to threshold differences; moving the lead closer to or further from the nerve resulted in large changes in threshold magnitude (FIG. 11C). Further differences in activation thresholds between contacts were attenuated when the lead was closer to the nerve, and accentuated when it was further from the nerve. Regardless of the distance to the nerve, these models showed a monotonic decrease in thresholds with more rostral contacts, which matches experimental results in sheep. This may be driven by the bone of the foramen restricting current flow away from the more rostral contacts, as well as the bend in the nerve increasing fiber excitability. When we moved the lead longitudinally (along the length of the nerve) within the foramen (FIG. 11 Error! Reference source not found.D), caudal shifts resulted in the contact thresholds becoming non-monotonic. A lack of threshold monotonicity may indicate a lead placed too deep. When we shifted the lead rostrally, we observed a large increase in thresholds on contact 3. Very high thresholds on the rostral contacts could indicate lead placement which is too shallow. Shifts and rotations transverse to the nerve had little effect on thresholds (FIG. 11E and FIG. 11F). Rotations towards and away from the nerve resulted in strong monotonic relationships between thresholds and the active contact, depending on which direction the lead was rotated. This suggests that thresholds could act as a proxy for determining the angle of the lead with respect to the nerve.

### Example 3

Lead Design-Increased Contact Spacing. Disclosed herein are lead designs that increase the effectiveness of sacral neuromodulation in treating symptoms and reduce side effects of stimulation.

Conventional lead design used for sacral neuromodulation has proven effective for many patients, however, novel designs could offer benefits including increased responder rate, increased efficacy, reduced side effects, and reduced battery consumption.

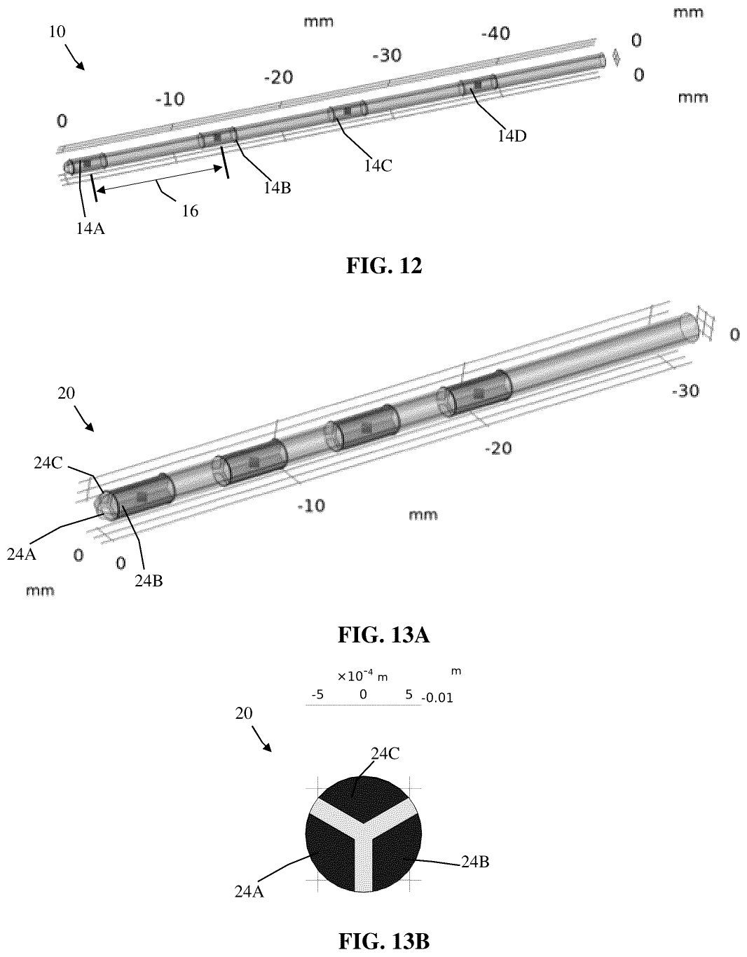

With reference to FIG. 12, an electrode is illustrated with increased contact spacing with the intention of determining the upper limit before thresholds begin to rise again (once the contacts are far enough, each will act like a monopole.

Embodiment of the present disclosure include an electrode 10 for stimulating a sacral nerve in a subject in need thereof, the electrode comprising a plurality of contacts 14A, 14B, 14C, 14D, wherein center-to-center spacing (e.g., spacing 16) between adjacent contacts is within a range of about 6 mm to about 18 mm.

In some embodiments, the electrode comprises a plurality of contacts, wherein the center-to-center spacing between adjacent contacts is within a range of about 12 mm to about 16 mm.

### Example 4

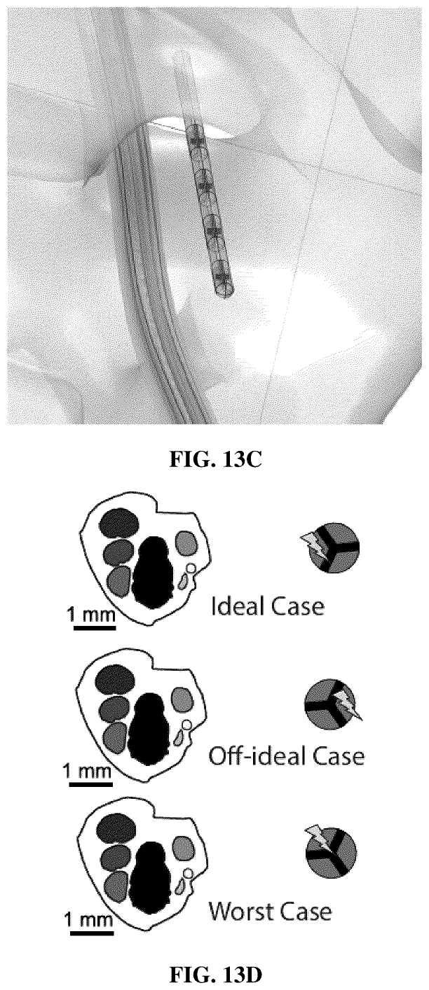

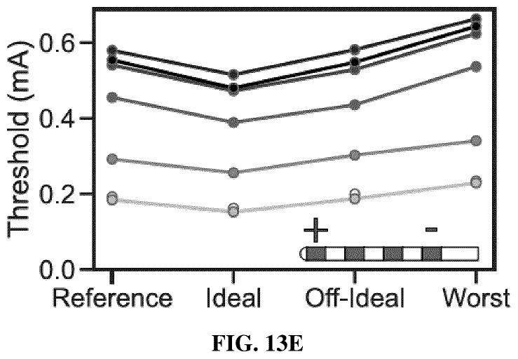

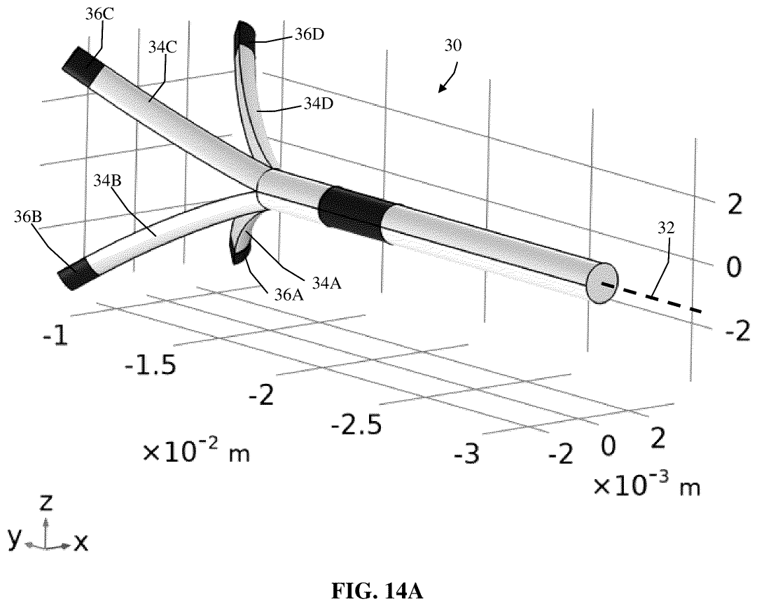

Lead Design-Segmented. With reference to FIGS. 13A-13E, we first tested a deep brain stimulation style segmented lead, splitting each contact into three contacts covering approximately 120 degrees. We instrumented the lead identically to the reference model, and rotated the lead so that, in one case, a row of contacts was pointed directly at the nerve, and in the other, the direction of the nerve was between two rows of contacts. For each row of contacts, we tested bipolar stimulation using the most rostral contact as the anode, and the most caudal contact as the cathode. While contact orientation around the lead had a small effect on absolute threshold values, there was no change in the selectivity of activation (fascicles activated in the same order regardless of orientation). Surprisingly, moving the lead closer eliminated the rotary benefit to overall threshold values, and again did not result in any changes in selectivity.

Embodiments of the present disclosure include an electrode 20 for stimulating a sacral nerve in a subject in need thereof, the electrode comprising: a plurality of circumferentially segmented contacts 24A, 24B, 24C.

In some embodiments, the electrode comprises at least three circumferentially segmented contacts.

### Example 5

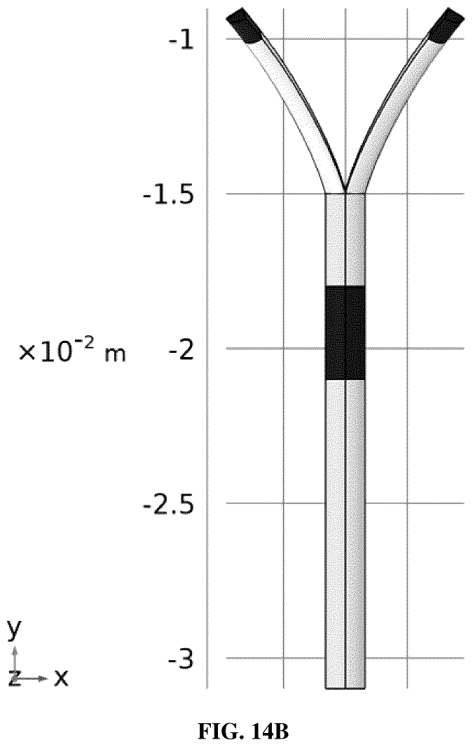

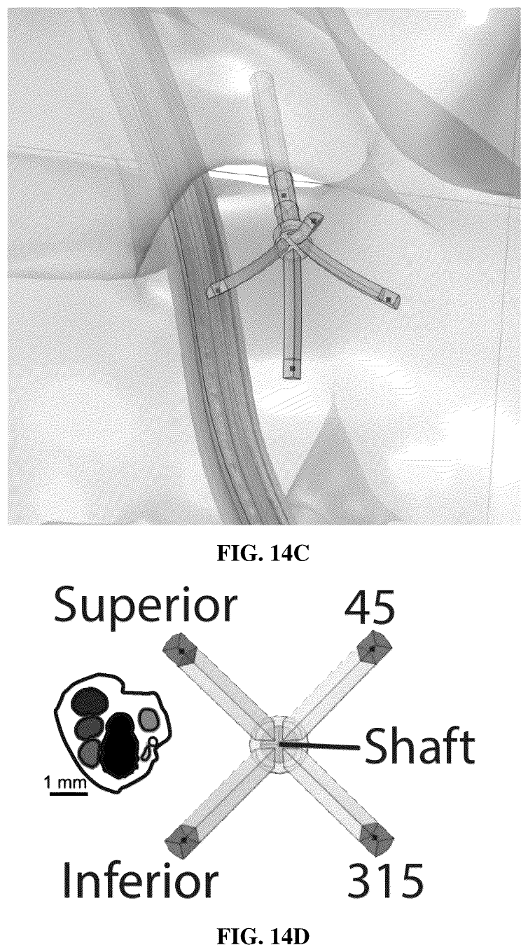

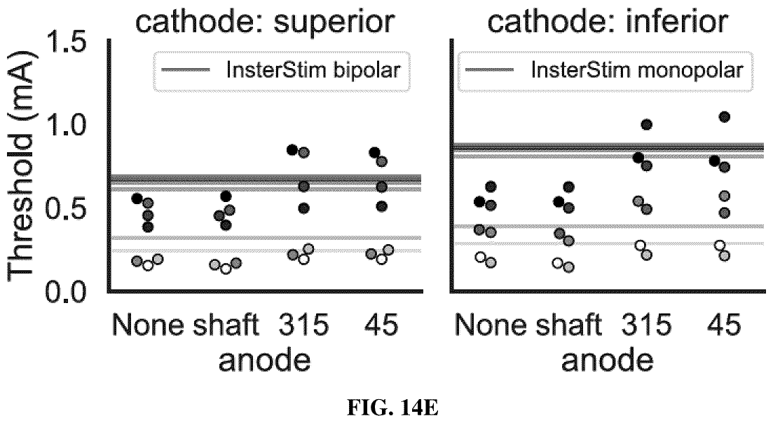

Lead Design-Umbrella. With reference to FIGS. 14A-14E, we tested an “umbrella” lead. We instrumented the lead in the same position as the interstim electrode. The contacts were positioned near the superior and inferior sides of the nerve, and were thus able to bias activation towards whatever side received cathodic stimulation. Surprisingly, even though the electrode fiber distance was greatly reduced, absolute threshold values were close to the results using the InterStim electrode.

Embodiments of the present disclosure include an electrode 30 for stimulating a sacral nerve in a subject in need thereof, the electrode comprising: a plurality of tines3 34A, 34B, 34C, 34D, wherein a contact 36A, 36B, 36C, 26D is positioned at a terminal end of each of the plurality of tines 34A-34D.

In some embodiments, the electrode comprises a plurality of tines 34A-34D extending radially away from a centerline 32 of the electrode 30, and each of the plurality of tines includes a plurality of contacts along a length of the tine.

### Example 6

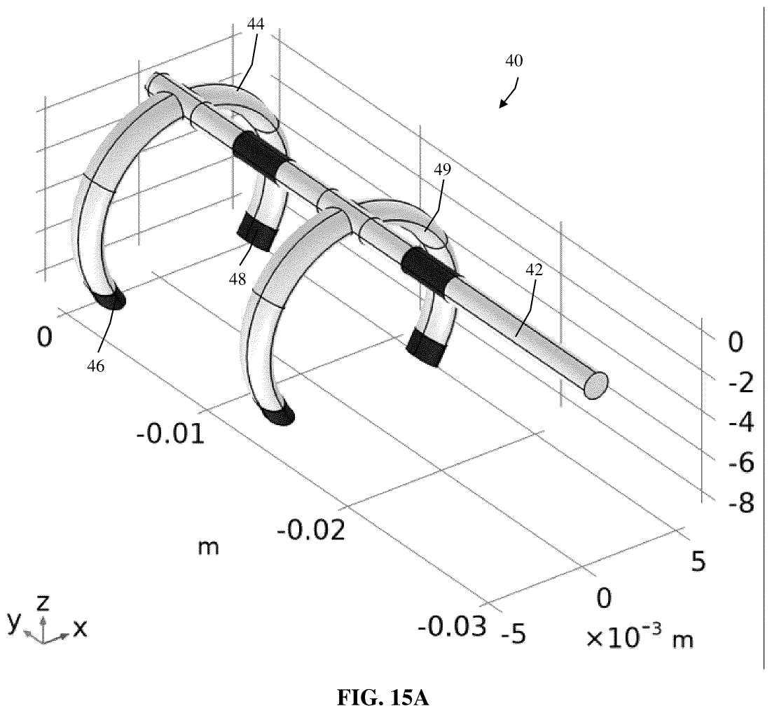

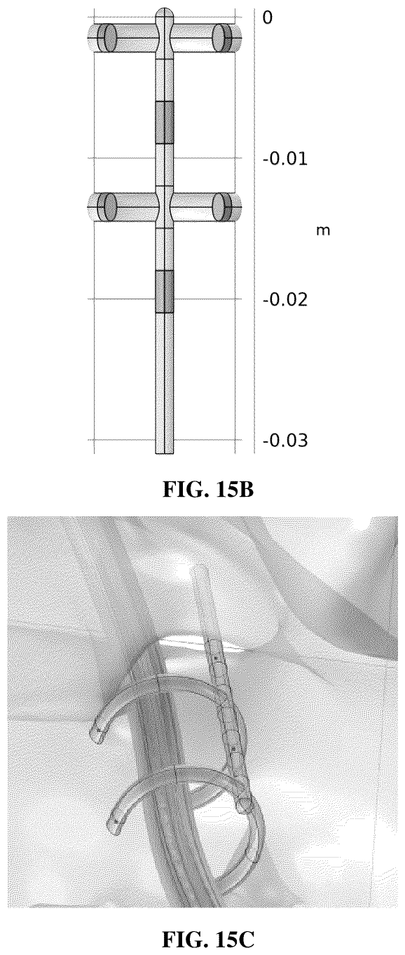

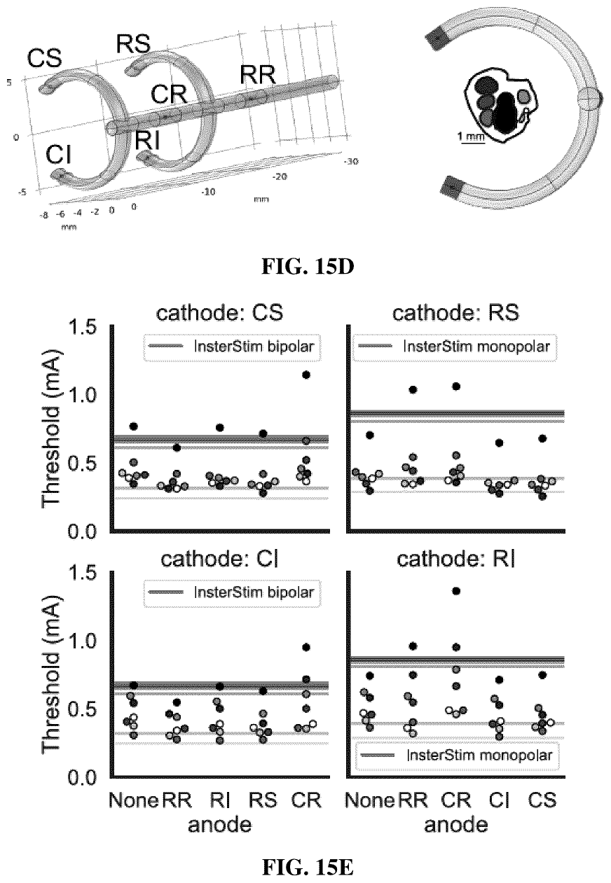

Lead Design-Loop. With reference to FIGS. 15A-15E, we tested S3 activation using a “loop” lead, with contacts deployed around the nerve. This lead was able to achieve fully selective activation for 4/7 fascicles, and was able to strongly manipulate fascicle selectivity ratios for all but the large central fascicle. Further, this lead was able to achieve activation threshold markedly lower than those obtained using the interstim.

Embodiments of the present disclosure include an electrode 40 for stimulating a sacral nerve in a subject in need thereof, the electrode comprising: a shaft 42 and a loop portion 44 with a first contact 46 at a first end of the loop portion and a second contact 48 at a second end of the loop portion.

In some embodiments, the electrode comprises a shaft and a loop portion extending from the shaft; wherein the loop portion includes a first contact at a first end of the loop portion, a second contact at a second end of the loop portion, and an intermediate contact positioned between the first contact and the second contact.

In some embodiments, the loop portion 44 is a first loop portion and the electrode further comprises a second loop portion 49 extending from the shaft 42.

In some embodiments, the 1 the first loop portion is positioned at a terminal end of the shaft and the second loop portion is axially spaced from the first loop portion along the shaft.

Embodiments of the present disclosure include an electrode for stimulating a sacral nerve in a subject in need thereof, the electrode comprising: a) a plurality of contacts, wherein center-to-center spacing between adjacent contacts is within a range of about 6 mm to about 18 mm; b) a plurality of circumferentially segmented contacts; c) a plurality of tines, wherein a contact is positioned at a terminal end of each of the plurality of tines; and/or d) a shaft and a loop portion with a first contact at a first end of the loop portion and a second contact at a second end of the loop portion.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of treating or preventing at least one symptom associated with a disease or condition in a subject in need thereof, the method comprising:
stimulating a sacral nerve in the subject with an electrode according to a set of stimulation parameters;
wherein the set of stimulation parameters is selected based on a three-dimensional model; wherein the three-dimensional model includes a pelvic organs model, a sacral nerve model, and an electrode model.

2. The method of claim 1, further comprising placing the electrode in a location suitable to deliver electrical stimulation to the sacral nerve.

3. The method of claim 2, wherein the location is at least partially determined from the three-dimensional model.

4. The method of claim 1, wherein the disease or condition comprises overactive bladder, urge urinary incontinence, urinary urgency frequency, underactive bladder, urinary retention, fecal incontinence, constipation, pelvic pain, and/or sexual dysfunction.

5. The method of claim 1, wherein the at least one symptom comprises urinary frequency, urinary urgency, nocturia, urge incontinence, feeling of not being empty, small voided volumes, straining to void, pain, or any combination thereof.

6. The method of claim 1, wherein the sacral nerve model includes a plurality of fascicles and epineurium based on histology nerve cross sections and/or imaging data.

7. The method of claim 1, wherein the three-dimensional model includes the electrode model positioned at least partially within a foramen in a sacrum of the pelvis model.

8. The method of claim 1, wherein the set of stimulation parameters includes one or more of: stimulation pulse amplitude, stimulation pulse duration, stimulation waveform shape, stimulation pulse repetition rate, and temporal pattern of stimulation pulses.

9. An electrode for stimulating a sacral nerve in a subject in need thereof, the electrode comprising:
a) a plurality of contacts, wherein center-to-center spacing between adjacent contacts is within a range of about 6 mm to about 18 mm;
b) a plurality of circumferentially segmented contacts;
c) a plurality of tines, wherein a contact is positioned at a terminal end of each of the plurality of tines; and/or
d) a shaft and a loop portion with a first contact at a first end of the loop portion and a second contact at a second end of the loop portion.

10. The electrode of claim 9, wherein the electrode comprises a plurality of contacts, wherein the center-to-center spacing between adjacent contacts is within a range of about 12 mm to about 16 mm.

11. The electrode of claim 9, wherein the electrode comprises at least three circumferentially segmented contacts.

12. The electrode of claim 9, wherein the electrode comprises a plurality of tines extending radially away from a centerline of the electrode, and each of the plurality of tines includes a plurality of contacts along a length of the tine.

13. The electrode of claim 9, wherein the electrode comprises a shaft and a loop portion extending from the shaft; wherein the loop portion includes a first contact at a first end of the loop portion, a second contact at a second end of the loop portion, and an intermediate contact positioned between the first contact and the second contact.

14. The electrode of claim 13, wherein the loop portion is a first loop portion and the electrode further comprises a second loop portion extending from the shaft.

15. The electrode of claim 14, wherein the first loop portion is positioned at a terminal end of the shaft and the second loop portion is axially spaced from the first loop portion along the shaft.

16. A method of selecting a desired lead position to treat or prevent at least one symptom associated with a disease or condition in a subject in need thereof, the method comprising:
generating a three-dimensional model of a pelvis and a sacral nerve based on imaging from a subject;
adding an electrode model to the three-dimensional model in a candidate position relative to the sacral nerve;
simulating neural activation of the sacral nerve in the three-dimensional model for the electrode model in the candidate position;
moving the electrode model in the three-dimensional model to an alternative position relative to the sacral nerve;
simulating neural activation of the sacral nerve in the three-dimensional model for the electrode model in the alternative position; and
selecting the desired lead position from one of the candidate position and the alternative position based on simulated neural activation.

17. The method of claim 16, further comprising:
adding an alternative electrode model to the three-dimensional model;
simulating neural activation of the sacral nerve in the three-dimensional model for the alternative electrode model in a plurality of positions relative to the sacral nerve; and
selecting a desired lead design from one of the electrode model and the alternative electrode model based on simulated neural activation.

18. The method of claim 16, wherein simulating neural activation of the sacral nerve in the three-dimensional model includes simulating a plurality of candidate lead configurations and determining activation of nerve fibers in a plurality of fascicles.

19. A method of selecting a desired lead configuration to treat or prevent at least one symptom associated with a disease or condition in a subject in need thereof, the method comprising:
generating a three-dimensional model of a pelvis and a sacral nerve based on imaging from a subject;
adding an electrode model to the three-dimensional model in a position relative to the sacral nerve; wherein the position is based on imaging from the subject;
simulating neural activation of the sacral nerve in the three-dimensional model for a plurality of candidate lead configurations of the electrode model; and
selecting the desired lead configuration from one of the plurality of candidate lead configurations based on simulated neural activation.

20. The method of claim 16, further comprising programming a pulse generator to deliver electrical stimulation to the subject based on the desired lead configuration.
